(19) United States
(12) Patent Application Publication
(10) Pub. No.: US 2006/0039899 A1
(43) Pub. Date: Feb. 23, 2006

(54) Animal nutritional product that increases weight gain and reduces diarrhea morbidity, mortality and severity by stimulation of natural immune response, nutritional support of immune function and supplemental nutricines and probiotics

(76) Inventor: Robert Terrell Winn, Big Sandy, TX (US)

Correspondence Address: Robert Terrell Winn DVM, 140 PR 3180, Big Sandy, TX, 75755, US

(21) Appl. No.: 10/923,313

(22) Filed: Aug. 23, 2004

(51) Int. Cl. A61K 39/02

(52) U.S. Cl. 424/115; 424/234.1

(57) ABSTRACT

A method of collecting, sterilizing, standardizing, drying and mixing rumen fluid while retaining the activity of the contained bacterial polysaccharides is described. Nutritional compositions including the resulting product, probiotics, nutricines, vitamins, minerals, an amino acid, and a monosaccharide are depicted. The use of this resulting nutritional composition the first few days of young animals' lives result in decreased diarrhea morbidity, severity and mortality. It also helps supply nutrients for the support of natural immune response and function.

### CROSS-REFERENCES

There are no related applications to be cross-referenced.

### FEDERALLY SPONSORED RESEARCH OR DEVELOPMENT

The development and research for this invention involved no federal or state funding. It was supported in full by private funding.

### Compact Discs and Electronic Data

There are no electronic data or compact discs included with this submission.

### DETAILED DESCRIPTION AND SPECIFICATION

### FIELD OF THE INVENTION

The present invention relates to the collection, processing, sterilization and stabilizing of rumen fluid in a dry form to preserve and standardize the bacterial polysaccharides contained therein. It further relates to the subsequent utilization of this product with a mixture of probiotics, nutricines, vitamins, minerals, an amino acid, and a monosaccharide. In particular this invention is fed to young animals for the first few days of life to increase weight gain, reduce diarrhea severity, morbidity and mortality by stimulation and support of the animals natural immune response.

### BACKGROUND OF THE INVENTION

Animals are raised in concentrated rearing units. These units are used on a constant basis resulting in a build up of contamination and disease organisms. The young newborn animals are frequently affected with diarrhea. Although management practices to maximize the passive immunity are used and sanitation measures followed to minimize the exposure of newborns to virulent organisms, the diarrheal disease process is the most costly disease process affecting the rearing of newborns.

There is both a political move and a public health concern with the use of antibiotics as feed additives. There are also public health concerns with the extra-label use of antibiotics in food producing animals. To maintain health and increase productivity without the use of antibiotics is the goal of many endeavors at this time (Donovan, D. C., et al, Growth and Health of Holstein Calves Fed Milk Replacers Supplemented with Antibiotics or Enteroguard, 2002, J. Dairy Sci. 85:947-950: Webb, P. R., et al, Addition of fructooligosaccharide (FOS) and sodium diacetate (SD) plus decoquinate (D) to milk replacer and starter grain fed to Holstein calves, 1992, J. Dairy Sci. Vol 75 Suppl. 1:300). As such, there are many studies and products, which attempt to increase the immuno-competence of the neonate. Vaccines, serum immunoglobulins, colostrum replacers and colostrum antibody preparations have all been used to improve the neonate's immune status. Other nutritional supplements have been described. U.S. Pat. No. 6,667,063 B2 describes a composition containing as the essential ingredients colostrum, a selected whey product and defined amounts of selenium or an organic or inorganic, water soluble selenium precursor. The goal, ingredients and method of action are different from the present composition.

Most of the ingredients of this composition product are currently commercially available. The combination is not currently commercially available and the resulting increase in weight gain, lowered number of treated animals and increased livability of the animals is surprisingly better than expected from the individual products alone. This type of response has been seen in ruminating animals as taught in U.S. Pat. No. 4,405,609. Most feed additives have to be fed for the full or at least an extended feeding period to achieve results. This composition product is only fed for the first 3-7 days of life and achieves results. Another composition is described in U.S. Pat. No. 6,258,399, which improves the immune system indirectly by providing early carbohydrate digestion. This increase in energy stimulates the growth stimulation hormones, which in turn starts the growth and response of the immune response system. Similarly, it is only fed for the first days of life with continued results and then the chicks are switched to the usual feed. The composition of the current patent contains bacterial polysaccharides, vitamins, organic minerals and probiotics, has a different effect on the animal by causing a localized immune response, and does not supply a disaccharide or an oligosaccharide as described in the composition of U.S. Pat. No. 6,258,399.

Another composition is described in U.S. Pat. No. 6,365,152 B1. This composition is used for the treatment of scours and not the prevention of scours. It contains many of the same nutrients found in the current patent. However, it claims the mix is a solution of trace mineral, which is mixed with other ingredients including kelp. We are taught that kelp is a natural source of carbohydrates, amino acid, vitamins, minerals and trace elements. We are further taught that kelp also contains over 60 minerals and elements including iodine, 21 amino acids, fiber, simple and complex carbohydrates. Kelp is added at the rate of 1 gram per treatment. There is no kelp in my current patent. Also, the current patent contains approximately 5 grams of the purified amino acid threonine per each daily ration for calves of the current composition, compared to the small amount that might be found in one gram of kelp. There are no bacterial polysaccharides included into the composition of U.S. Pat. No. 6,365,152 B1. Also, the referenced patent is for treatment of scours in farm animals, not for the prevention of scours and increased weight gain. A second composition for the treatment of scours contains three energy sources and is described in U.S. Pat. No. 6,066,341.

One of the objectives of this invention is to provide nutrients that increase the ability of the young animal to develop its own immunity. B complex vitamins, and organic trace minerals are added to the formulation to insure that all enzymatic activity may occur without compromise due to a deficiency of catalytic enzymes. Due to the expense of these products, they are not added to milk replacers used in rearing calves. These products do not occur in whole milk from the dam of mammals in sufficient levels without supplementation. Due to the low dry feed intake the first few days of life, it is unlikely that young mammals eat sufficient feed to obtain the recommended level of intake of these nutrients for optimum production.

These vitamins and minerals may be added to diets of young chicks but due to the inanition or lowered dry feed intake the first few days of life, it is unlikely that sufficient feed is eaten to obtain the recommended level of intake for optimum production. As taught in U.S. Pat. No. 6,733,759 B2, a specialized method of feeding the newborn chicks must be used. The ingredients used in this product may be administered to poultry by using the unique delivery system described in the aforementioned patent or a similarly devised delivery system.

A second objective of this invention is to provide specially prepared and selected probiotics that can be used to help keep the flora of the gut populated with bacteria that support health of the gut and over grow or suppress the virulent bacteria. Methods of collection and culture of these specially derived probiotics are described in U.S. Pat. Nos. 4,689,226; 6,214,335 B1; and 6,645,530 B1. Continuously fed probiotics have been shown to improve body weight gain, teed conversion, and fecal condition of newborns (Abe, F., N. Ishibashi and S. Shimamura, Effect of Administration of Bifidobacteria and Lactic Acid Bacteria to Newborn Calves and Piglets, 1995, J. Dairy Sci. 78:2838-2846). These products are available as dry feed inclusions and also as individual innoculants to be administered orally on a daily basis as described in U.S. Pat. Nos. 4,985,246; 5,718,894 and 5,902,578. Probiotics are added to other treatment packages currently available, but are not commonly added to powdered milk replacers nor are seldom used as additives in whole milk fed to calves. They may be added to diets of young chicks or pigs, but due to the low dry feed intake the first few days of life, it is unlikely that sufficient feed is eaten to obtain the recommended level of intake for optimum inoculation. Another method of inoculation of probiotics into chicks is by spraying a suspension of viable microorganisms of lactic acid bacteria on newborn chicks within 4 days of hatching, U.S. Pat. No. 6,410,016 B2. By adding them to the composition, a more complete stimulant package is formed, and specific strains known to be advantageous to the specific species of animal being fed may be used. In most situations, only one probiotic mixture/package is available in a feed mill and this is used in the feed produced regardless of the species for which the feed is intended.

U.S. Pat. No. 5,374,425 describes the manufacture of a killed probiotic. The stabilization process is somewhat similar to the process used in the current invention. Both products are autoclaved to kill the bacterial cells. In the current invention, autoclaving takes place at 116° C. for 45-60 minutes at a pressure of 10 p. s. i. The referenced patent uses a variable temperature (100° to 121° C.) and a shorter duration (15-30 minutes). Also there is also a difference in drying. To separate the bacteria cells in U.S. Pat. No. 5,374,425 a flocculating agent is added to the culture and the cells are allowed to settle out. The liquid is decanted off. Heat, spray or freeze-drying is promoted as acceptable drying methods and the use of a drying agent is proposed. These methods except for freeze-drying are not acceptable in the current invention. Another difference is that the current patent uses rumen fluid bacteria, while this patent uses a specific culture or mixes of dried specific cultures. U.S. Pat. No. 4,021,303 also produces killed organisms. This process includes chemically treating the microorganisms with alkali at a pH of 10.5-12.9 and a temperature of 0°-30° C., washing with water and mechanically rupturing the bacteria at a pH of 7-10.2.

Another aim of this invention is to increase the natural local immune response by the exposure of the gut to bacterial polysaccharides in a measured, safe and controlled manner. Rumen fluid has been shown to increase growth rate in calves, decrease morbidity, mortality and use of treatments for diarrheal disease (Muscato, T. V., L. O. Tedeschi, and j. B. Russell, The Effect of Ruminal Fluid Preparations on the Growth and Health of Newborn, Milk-Fed Dairy Calves, 2002, J. Dairy Sci., 85:648-656). Rumen fluid has been shown to contain bacterial polysaccharides. These bacterial polysaccharides are considered the “active ingredient” in rumen fluid. Bacterial polysaccharides nave been shown to elicit localized immunity. Rumen bacteria have been reported to have extracellular polysaccharide “coats” that are similar to those found on many Gram (−) organisms (Costerton, j. W., H. N. Damgaard and J. K Cheng, Cell envelope morphology of rumen bacteria, 1974, J. of Bacteriology, 118:1132-1143). It is my belief that this similarity is the reason ruminal fluid bacteria are the best to use for this desired result.

We are taught in U.S. Pat. No. 6,444,210 B1 that bacterial polysaccharides have been used as vaccines to enhance specific humoral immunity and in the particular invention named they are used to enhance general cellular immunity against a wide variety of microorganisms. The mentioned patent describes a method of isolation, purification, stabilizing and using Brucella abortus and Yersinia enterocoiltica outer polysaccharide as an immunizing agent. This differs from the current invention in that the current invention makes no strides toward selecting, isolating or purifying a particular polysaccharide considered effective as an immune modulator. It further differs from the current invention in that the current invention makes no effort toward selecting, isolating or purifying the bacterial polysaccharide from the rest of the ingredients in the rumen fluid, except for excluding physically large fibers and particles. Also, the number of species of bacteria in the rumen is great and there are no steps taken to reduce this number of species. Three other similar claims have been made for specific extracts of polysaccharides to be used as vaccinal agents, see U.S. Pat. Nos. 4,210,641; 6,007,818; and 6,045,805. The current invention differs from these three inventions for the aforementioned reasons.

We are told in U.S. Pat. No. 6,087,342 that the extraction of polysaccharides that have immune stimulating properties results in small fragments of the longer chain immune-stimulating polysaccharides. These fragments that occur have lower bioactivity than that found in the parent substance. This patent involves the use of a special substrate to bind the small fragments to which potentates the activity of the fragments. This differs from the current invention in two main aspects. First an isolated product in the form of bacterial polysaccharides or bacterial nucleic acids from bacteria is used. Second this is bound to a specialized substrate. My invention uses the whole rumen fluid, or the whole bacterial culture, as it were. I also use the rumen ingesta smaller than 2 mm as the substrate that is used to carry the bacteria.

Another novel method of stimulating the immune system with bacterial produced products is described in U.S. Pat. No. 5,840,318. This method consists of growing bacteria in a stressed manner to increase the stress response factors production of the bacteria. These products are then isolated and used to activate and modulate circulating macrophages. This differs from the current invention in several methods, but primarily due to the fact that the bacteria are stressed instead of grown to peak growth rates. The stress response factors desired by the described method are not a consideration in the current invention.

Bacterial polysaccharides are produced under several patents for use as food thickeners. These patents use bacteria of the genus Xanthomonas and describe a process to grow the bacteria using specialized media or growing conditions.

These descriptions are found in U.S. Pat. Nos. 3,328,262; 3,391,061; 3,433,708; and 4,692,408. Other bacterial polysaccharides are produced for use as viscosity regulators used in various manufacturing processes as described in U.S. Pat. No. 4,567,140.

Rumen fluid fed fresh has resulted in increased growth rate in calves, decreased morbidity, mortality and use of treatments for diarrheal disease (Muscato, T. V., L. O. Tedeschi, and J. B. Russell, The Effect of Ruminal Fluid Preparations on the Growth and Health of Newborn, Milk-Fed Dairy Calves, 2002, J. Dairy Sci., 85:648-656). The obvious problems to using fresh rumen fluid are the daily collection of the fluid. The chance of spreading disease. The need to maintain a fistulated animal on each farm. Rumen fluid may be sterilized and bottled to increase storage time. However, upon opening, the bottle must be refrigerated. Also, each farm would need to maintain the equipment to sterilize the rumen fluid.

Another problem is that there is no way to accurately measure the bacterial polysaccharide content of the rumen fluid daily on the farm. It has been shown that the number of rumen bacteria are affected by time of day, diet, time following feeding, location of sampling and diet physical characteristics (Bryant, M. P., and I. M. Robinson, Effects of Diet, Time After Feeding and Position Sampled on Numbers of viable Bacteria in the Bovine Rumen, 1968, J. Dairy Sci., 51:1950-1955; Bryant, M. P., and I. M. Robinson, An improved Nonseiective Culture Media for Ruminal Bacteria and its use in Determining Diurnal Variation in Numbers of Bacteria in the Rumen, 1961, J. Dairy Sci., 44:1446-1456). The result is a varying level of rumen bacterial polysaccharide content collected. This phenomenon was observed by other workers (Muscato, I. V., L. O. Tedeschi, and J. B. Russell, The Effect of Ruminal Fluid Preparations on the Growth and Health of Newborn, Milk-Fed Dairy Calves: 2002, J. Dairy Sci., 85:648-656).

The process of the current invention allows for the collection of rumen fluid; sterilization of the fluid to prevent disease spread; maximization of the bacterial polysaccharide in the fluid collected by proper timing of feeding and collection, and by specialized ration formulations to increase bacterial growth in the rumen; measurement of bacterial polysaccharide content; and standardization of the amount of bacterial polysaccharide used. There are patents used to measure the level of microbial activity and the presence of live organisms in fluid, however there is not a measurement to standardize the bacterial polysaccharides in a product for dosing. For examples see U.S. Pat. Nos. 5,970,163; 6,051,394; and 6,344,332 B1.

One of the main objectives of this process is to produce as large a population as possible and harvest them during peak concentration. There are many patents that deal with growing bacteria. The major difference found in the current invention is the use of a cow as an apparatus for the growth of bacteria. Also instead of liquid purified substrates this invention uses standard cow feeds as a substrate to produce the bacteria growth. The cow's rumen is considered to be an anaerobic growing situation. U.S. Pat. Nos. 3,002,894; 4,752,564; and 5,660,977 all deal with aerobic bacterial growth. Several of the patents deal with culturing processes that in some way control the growth of the culture. U.S. Pat. Nos. 4,021,304; 5,017,479; and 6,284,453 are all in this category but no apparatus is claimed in the patent. An apparatus is claimed in U.S. Pat. Nos. 2,686,754; 2,767,118; 3,010,881; 3,018,224; 3,227,557; 3,672,953; 3,766,010; 3,767,534; 3,880,716; 4,167,450; 4,230,806; 4,865,969; 4,900,669; 5,316,905; 5,541,056; and 6,716,617 B1.

U.S. Pat. No. 4,228,275 describes a process of producing a nitrogen containing polysaccharide. This process includes the use of a specific bacterium, not many species of bacteria as is found in the rumen. It also requires reaction with and aqueous ammoniacal solution at a temperature of 100° to 250° C. The resulting product is used to control viruses in plants. U.S. Pat. No. 4,529,701 describes a method of stimulating bacterial growth in an anaerobic digestion system. It specifically deals with improving digestion in sewage digestion systems that have gone sour and uses a mixture of an inhibitory ion regulation component and an inorganic pyrophosphate-containing compound.

This is not the first process to take advantage of products produced by microorganisms. I would draw your attention to some patented processes that may on first glance appear similar to this process. In U.S. Pat. No. 6,255,080 B1 rumen bacteria of the Butyrivibrio spp. are used to produce proteinaceous antibiotics that are resistant to gastric proteases, exhibit a high level of hydrophobicity, and are effective under anaerobic conditions. The Butyrivibrio spp. are isolated and cultured and screened for their production of bacteriocin-like activity. In U.S. Pat. No. 1,818,781 mixed cultures of bacteria were used to cause specialized fermentation to produce ethyl alcohol, lactic acid, butyric acid, butyl alcohol, isopropyl alcohol, acetone, etc. Neither patent uses the same growth media, apparatus nor obtain the same end product as the current application.

An important goal of this invention is to make the product available in an easily storable, transportable and usable form. Dried rumen fluid was considered not to have the activity of liquid rumen fluid and therefore not to be a viable alternative (Dr. J. Russell, Personal communication, Aug. 6, 2002). Field trials by the inventor using warm forced air-dried rumen fluid on drying aids have not to given the beneficial results obtained with sterilized liquid rumen fluid. Field trials by the inventor with freeze dried sterilized rumen fluid have been shown to give results equal to those obtained with sterilized liquid rumen fluid. This gives this invention the distinct advantages of being easily stored, transported, used and standardized for bacterial polysaccharides. In U.S. Pat. Nos. 4,855,149 and 4,877,634 a method of drying bacterial polysaccharides produced from a culture of Leuconostoc is described. The process includes using drying aids and preferably spray drying or drying “in any manner”. The end product is to be used as quality improvers (e.g. texture, stability or thickness) for foods. U.S. Pat. No. 4,391,887 describes a method of drying mixed cultures of bacteria to maintain the bacterial activity. This process differs in that live cultures are stored to be used as inoculates for the degradation of industrial organic effluents.

An additional intention of this invention is to help the immune system by supplying extra antioxidants for the first few days of life to help build up the body stores. Vitamins A and E in addition to the trace minerals copper, zinc and selenium are all important antioxidants. As an additional source of these nutrients, this product allows for the stores of these nutrients to be built up.

Another objective of this invention is to provide an alternate supply of the amino acid threonine for the first few days of life. This is to insure that the diet is not deficient in the amino acid that is most needed in the formation of mucous in the gut. This is an additional advantage in that this amino acid is not specifically added to dry feeds for young animals nor is if found in high levels in whole milk or milk replacer.

A final goal of this invention is to use only AFFCO approved products.

This product may be administered orally to individual animals by either drenching or dosing with a solution of the product. It may be fed to individual animals by mixing it into the milk fed to that animal. It may be fed by some unique system as mentioned above for poultry. Or, it may be top-dressed on dry feed for swine and poultry. The feeding period will range from 3-7 days and should start on day 1 or 2 of life.

### SUMMARY OF THE INVENTION

A process of producing, collecting, processing, stablizing and drying rumen bacterial polysaccharides. These dried rumen bacterial polysaccharides are included into a novel composition with vitamins, probiotics, minerals, an amino acid and monosaccharide are used to reduce the effect of diarrhea in young animals. The present invention when used for the first few days of life is capable of reducing the severity, incidence and mortality from diarrhea. It also results in increased body weight gain.

### DRAWINGS

There are no drawing

### SPECIFICATION

The invention Animal Nutritional Product that increases Weight Gain and Reduces Diarrhea Morbidity, Mortality and Severity by Stimulation of Natural immune Response, Nutritional Support of immune Function and Supplemental Nutricines and Probiotics is actually a method, process and composition of matter. The first section will be the process by which rumen fluid is harvested to protect and preserve the bacterial polysaccharides within it, in a manner that is easily mixed, standardized, stored, transported, measured and re-hydrated.

Process

The animals used for this process must be selected to be of sufficient size to support removal of rumen fluid from a rumen cannula that is placed into surgically created rumen fistulas in their left flank region. This is also important in that the size of cannula needed for daily withdrawals of fluid is a four-inch (4″) cannula. If a smaller animal is used, the fistula and the cannula may be below the fluid line in the rumen. This would allow for constant seepage of rumen fluid and irritation to the area of the rumen fistula. This would severely limit the length of time of collection that would be possible in the donor animal. This would also drastically affect the health of the fistulated animals. The animals should be of a temperament to allow daily man to animal contact, manipulation, handling and care with the least of actual physical restraint.

The animals should be tested for Johne's disease, TB, and BVD. Brucellosis testing or vaccination is recommended. Vaccination with IBR, PI3, BVD and BRSV are recommended. Also the Clostridial diseases and Leptospirosis should be included in the vaccination program. Parasite control should be administered as needed.

The surgical preparation and placement of the rumen fistula should be done a minimum of three (3) weeks prior to actual time of onset of collection. This will allow for healing of the incision site, removal of the sutures, and placement of the final cannula for long-term care and collection. The site for fistula insertion should be high on the left flank, but not so high that the cannula will be above the transverse processes on the outside of the animal, nor be forced to bend under the processes on the inside of the rumen. This will allow for a minimum of irritation from the cannula itself. This is paramount in keeping the donor animal healthy. As mentioned above, the bottom of the fistula should be above the fluid line in the rumen to prevent constant seepage. The surgical fistula should be round, that is, the same shape of the cannula. It should be four-inches (4″) in diameter when cut. A smaller cannula (three-inch, 3″) should be placed into the fistula following surgery to allow healing. This can be replaced at two to three weeks following surgery and removal of the sutures. The placement of the final four-inch (4″) cannula should not be done until the entire suture line is healed. If a smaller cannula is not placed into the fistula, the fistula will contract during the healing process and will prevent the use of the larger permanent cannula later (info@bardiamond.com, Cannulae Surgery Information; P. L. Rohwer, Personal communication, Jan. 2, 2003).

The animal should be placed on a special diet to increase the level of rumen bacteria. This should be done a minimum of two (2) weeks prior to the actual time of onset of rumen fluid collection. This portion of the process will allow the rumen bacteria to adjust to the new feed and increase in numbers and growth rate. This will also allow the rumen contents to gain the actual texture necessary to allow fluid collection without having to either remove dry ingesta, add water to the rumen fluid or remove the ingesta and rinse it with water to “wash out” the bacteria. The diet formulation, physical size and feeding regularity are all important in regulation of the rumen mat, which in turn affects the ease of collection of rumen fluid and the growth of bacteria.

Feeding should be done a minimum of two times a day. The timing of the feedings should be such that the major feeding period is done 12 hours prior to collection and a secondary feeding with grain done 4-8 hours prior to collection. The feedings should be coincided such that the collections may be made between 1100 and 1500 hours. This timing results in a near peak production of rumen bacteria (Bryant, M. P., and I. M. Robinson, Effects of Diet, Time After Feeding and Position Sampled on Numbers of Viable Bacteria in the Bovine Rumen, 1968, J. Dairy Sci., 51:1950-1955; Bryant, M. P., and 1. M. Robinson, An Improved Nonselective Culture Media for Ruminal Bacteria and its use in Determining Diurnal Variation in Numbers of Bacteria in the Rumen, 1961, J. Dairy Sci., 44:1446-1456). Multiple feedings keep the bacterial mass growing and near the peak concentration, thereby allowing for larger collections of bacterial polysaccharides. Multiple feedings also prevent large swings in bacterial growth that might influence the types of bacterial available. It is important that multiple species are available for collection to give the widest range of possible bacterial polysaccharide attachments for the neonate's gut to recognize and to subsequently develop immunity against.

The total amount collected is approximately one to one-and-one/half (1-1½) gallons of rumen fluid per collection period. A collection period is defined as the actual collection of fluid ingesta by withdrawal of fluid through a collection tube. The collection is made by first removing the cap on the rumen cannula. The hose is introduced into the rumen by first “clearing” a path with a sleeved arm. This accomplishes two goals. One is to allow the passage of the collecting hose through the rumen mat so that it may reach the fluid level. The second is to free up liquid in the mat that contains a higher level of bacteria. The hose end is covered with your hand when introducing into the rumen down to the level of the rumen fluid. The hose used may be solid or have small holes in the wall in the last six-inches (6″) of the inserted end. Vacuum or siphoning is then used to withdraw the fluid.

A maximum of one collection period per day is performed. This allows the donor animal time to readjust her rumen fluid level, consume more feed to replenish what has been removed, and to readjust her electrolytes that may be affected by removing rumen fluid without the lower gut having the chance to regain nutrients that are contained within.

Once the fluid is removed, it should be collected into a stainless steel, glass or specially designed hard plastic receptacle to prevent any reaction between the fluid and the receptacle. The receptacle should be clean, disinfected or sterilized and rinsed with de-ionized or distilled water. This physical composition requirement and cleaning methodology will be the same for all of the numerous processing receptacles and utensils.

The fluid is then sieved through a series of sieves, starting with the largest size holes first and progressing to the smallest. The final size sieve should have holes a maximum of two (2) millimeters in diameter. The final solution will contain a slight amount of sediment. The solution should be mixed thoroughly enough to suspend this sediment and then metered into containers for autoclaving. The mixing process must be constant during filling of containers for autoclaving or the container must hold the total collection, or mixture of collections. This step is necessary to allow for testing and standardization of the bacterial polysaccharide in the final dried product.

The autoclaving process should be started immediately to prevent excess gas formation within the container that will prevent the sealing of the container. Allowing the fluid to incubate for a period of time prior to autoclaving will increase the bacterial population, however, it may also change the population (Wells, J. E. and J. B. Russell, Why Do Many Ruminal Bacteria Die and Lyse so Quickly?, 1996, J. Dairy Sci. 79:1487-1495). This probable change in population has not been studied nor the results of the resulting product tested. Although this step is contemplated and planned, until this is done, I feel that this is a major step in controlling the quality and standardization of the product.

These collections are labeled to allow control of each collected lot. Each lot collected must be tested for bacterial polysaccharides. Therefore it is important to keep each lot identified the same. In addition, each lot autoclaved must be tested for bacterial growth, both aerobic and anaerobic. It then becomes necessary that each lot autoclaved is identified, regardless of the collection lot it originated. Autoclaving should be done for a period of 45-60 minutes at of temperature of 240° F. (116° C.) and 10 pounds per square inch of pressure.

Following autoclaving, the rumen fluid collection is allowed to cool. Freeze-drying may be started immediately or it may be stored for variable amounts of time prior to further processing. Prior to freeze-drying, samples from each collection lot must be taken to be tested for bacterial polysaccharides. In addition, each lot autoclaved must be sampled and tested for bacterial growth, both aerobic and anaerobic growth. These samples must be taken prior to freeze-drying, but the testing does not have to be finished prior to freeze-drying. Each lot of freeze-dried material must be labeled with the collection lot and the autoclaved lot. The amount of material from each collection lot placed into each freeze-drying tray or lot must be recorded.

The trays are first frozen in a not frost-free freezer and then placed into the freeze-drying chamber. The freeze-drying process followed is in the following schedule. It is probable and expected that the schedule will change from one type of freeze-drying equipment to another. In addition the depth of the trays and the amount of liquid used will also affect the drying time and results.

| Day | Set Point | Vacuum millitors |
|---|---|---|
| 1 | (−) 20° F. | 103 |
| 2 | (−) 15° F. | 104 |
| 3 | (−) 10° F. | 106 |
| 4 | (−) 5° F. | 99 |
| 5 | 0 | 102 |
| 6 | 10° F. | 87 |
| 7 | 20° F. | 90 |
| 8 | 30° F. | 94 |
| 9 | 40° F. | 92 |
| 10 | 50° F. | 91 |
| 11 | 70° F. | 92 |

Once the product is dried, it is removed from the chamber, scraped out of the drying tray into a mixing container. At this point it is ready to be mixed into an amino acid carrier that will be used to dilute the dried rumen fluid and allow it to be standardized in the invention.

Method

The invention Animal Nutritional Product that Increases Weight Gain and Reduces Diarrhea Morbidity, Mortality and Severity by Stimulation of Natural Immune Response, Nutritional Support of Immune Function and Supplemental Nutricines and Probiotics as stated above is actually a method, process and composition of matter. The process of producing the raw material used in the production of the final composition of matter has been described. A description of the Method of Standardization of the product will be described. Without a method of standardizing the bacterial polysaccharides, there would be no way of producing a product that works consistently and remains the same from batch to batch.

Method of Standardization

Each lot collected must be tested for bacterial polysaccharides. This test will quantify the amount of bacterial polysaccharides in the lot. The amount of product that is placed into the freeze dryer may then be mathematically used to determine the amount of bacterial polysaccharides found in the lot. This dried material will then be added to an amino acid carrier to allow for standardization.

For example, if the fluid prior to drying contained 300 μg hexose equivalent bacterial polysaccharides/ml. and there were 4000 ml. dried, we simply multiply the 4000 ml.×300 μg. This would equal 1,200,000 μg of bacterial polysaccharides in the total wafer of dried material. There must be two assumptions to continue. First, how much bacterial polysaccharide is desired per dose, and second, how large is the dose of carrier material to be used? I want to use 2,400 ug of bacterial polysaccharides per dose and I would like a 5-gram inclusion per dose. This batch of dried material contains 1,200,000 ug of bacterial polysaccharides. To determine the number of doses that may be made from this batch, divide the total μg of bacterial polysaccharides by the μg of bacterial polysaccharides desired per dose. 1,200,000 μg/2,400 μg per dose=500 doses. This is the number of doses we know we can make from this mix.

From previous experiments, 4000 ml of solution will render about 80 grams of dried product. The assumption was that there would be a 5-gram inclusion per dose. It is determined that this batch will make 500 doses. 500 doses×5 grams=2,500 grams. Now, put all of the dried material into a receptacle on a scale. The receptacle should have had the tare checked prior to the dried material being placed into it. Q. S. the total with the carrier amino acid to reach 2,500 grams net weight. This material should then be thoroughly mixed. This mixed product of the carrier amino acid and the dried rumen fluid will now be standardized and may be incorporated into the rest of the mix simply by weight.

Composition of Matter

The invention Animal Nutritional Product that Increases Weight Gain and Reduces Diarrhea Morbidity, Mortality and Severity by Stimulation of Natural Immune Response, Nutritional Support of Immune Function and Supplemental Nutricines and Probiotics is also a new composition of matter. It is not a composition in the sense of a new chemical entity, but in the sense of the same molecular structures only found in liquids but now available in a dry form that is fully functional. This of course refers to the freeze-dried bacterial polysaccharide component of the product.

Also the actual formula that includes this bacterial polysaccharide along with several other specific ingredients that add to the effectiveness of the bacterial polysaccharide and the overall product results in the complete product being a new chemical composition of matter. The formula includes a specific amino acid that supports the production of mucous found in the intestinal tract. It also contains monosaccharides, which are used to standardize the dose and supply energy without affecting the bacterial polysaccharides. Immunosupportive antioxidant products such as vitamins A and E, trace-minerals such as copper, zinc, manganese and selenium are added to the composition. B-complex vitamins to ensure proper enzyme function are added. Probiotics such as Lactobacilli and Bifidobacter are added to help establish a healthy population of bacteria in the gut as well as suppress the level of pathogenic bacteria in the gut.

Experimental Supporting Trials

Field trials with this mixture included with the freeze dried bacterial polysaccharide resulted in improved growth rate and weight gain over the use of the bacterial polysaccharide alone. Use of the specially collected rumen fluid bacterial polysaccharide resulted in less sick animals, less mortality and fewer treatments required in calves.

Trials

New Mexico Calf Treatment Trial

The objective of this study was to compare 3 different treatments for calves. The main exercise here was to find if freeze-drying was an acceptable treatment for the autoclaved rumen fluid. To ensure that each treatment was randomly assigned the treatment was assigned to the calves in the order they were delivered to the calf raiser. Both bull calves and heifer calves were treated. Each calf was assigned to the treatment group according to the order of delivery to the calf raising facility, the farm of origin and the sex of the calf. Bull calves derived from other farm(s) than C_Dairy were considered a separate subgroup. Each calf was assigned to the treatment group according to the color of the treatment that was next in the rotation. The rotation was determined to be white, green and red. There were 3 subgroups in the study: C_Dairy heifers, C_Dairy bulls and other dairies' bulls. The rotation of treatments was made within each of the subgroups. For example: Two heifers are delivered on Monday. The first is assigned to the white treatment, the second is assigned to the green treatment. The first bull delivered from C_Dairy is assigned to the White treatment. The first bull from other dairies is assigned to the White treatment. On Tuesday, four more heifers are delivered. The first is assigned to the red treatment, then white, green and red. The same type of rotation was used for C_Dairy bulls and other dairies' bulls. The C_Dairy bulls were separated from the other bulls for two reasons. First there were records available from C_Dairy on dam age and colostrum administration. Second, the other bull calves were assimilated from several other dairies and owned by the calf raisers instead of C_Dairy.

The calf raisers recorded the calf's dam's number (when available) and birth date (delivery date was considered acceptable). They also recorded which treatment the calf was assigned to. If available, they were asked to check the appropriate space if the calf was a twin or if the cow had to be helped to deliver the calf (the calf was pulled). The calf should be weighed on arrival. Colored grease markers were used to mark each pen to allow the workers the ability to quickly identify the treatment group the calves are assigned to.

The treatment assigned was given for seven days. The calves were treated only 1 time per day in the morning. The calf was to receive colostrum the first day and then receive the treatment for 7 days. The medicines used for each treatment group were:

Treatment—White Calf Treatment Group—White Powder Treatment—Freeze dried autoclaved rumen fluid with probiotics, chelated trace minerals, amino acids. Positive control—Green Calf Treatment Group—Green Liquid Treatment—

Autoclaved liquid rumen fluid colored with cake coloring.

Negative Control—Red Calf Treatment Group—Red Powder Treatment—Milk powder colored with Kool-Aid®.

The mixing and feeding instructions given to the calf feeders were:

Mix the treatment in the milk prior to feeding the calf. The treatment may be mixed for several calves at once, however it may tend to settle out if allowed to stand. The bottles should be filled immediately after mixing the treatment and then inverted once or twice prior to feeding. If the milk has to stand in a five-gallon container following mixing prior to feeding or pouring into bottles, remix the container prior to pouring up for the calves. Once mixed the milk will have a color the same as the treatment group. Pink milk to the calves with a red marked pen, white milk (yellowish-gray color) to the calves with white marked pen and green milk to calves with a green marked pen.

The powder treatment is mixed at 2 level teaspoons (tsp—small spoon) per bottle. When mixing for several calves, mix ¼ cup rounded plus two tablespoons level per 5-gallon bucket.

The liquid treatment is mixed at the rate of 8 cc per bottle or 80 cc per 5-gallon bucket. Shake well before drawing out this treatment. A needle is not needed to draw it out of the bottle. The tops have slits that will allow a syringe tip to be inserted to facilitate drawing out the treatment.

The monitoring instructions used during this trial are as follows:

Although the treatment is only given for seven days, the effects are expected to last until weaning. The calves should be monitored daily until weaning. At weaning the calves should be weighed and the weight recorded on the sheet containing the calf's birth date and dam #.

Should any of the animals become sick, treat them, as is your normal practice and record the date and the medicaments used.

Daily—Record any calves that are sick, and the medicines administered.

Results: The weight gains were better for the treated animals in two of the trial groups. The group of heifers did not show the same response. The difference in the incoming weight of the three treatment groups within the heifer group may have contributed to this lack of response. The difference in the gain between the treatment group and the average of the two control groups as shown below is 6.3 #, 6.2 # and 0.9# respectively. Due to irregularities in the recording of illnesses and differences in the treatments used between groups (C_Dairy vs Purchased) these data were not included into the analysis.

New Mexico Calf Trial

| | Number of | | | |
|---|---|---|---|---|
| | Calves in | In Weight | Out Weight | Gain During |
| Treatment | Group | in Pounds | in Pounds | Trial |
| PBG | 17 | 88.4 | 150.6 | 61.2 |
| PBR | 15 | 93.9 | 157.9 | 64.0 |
| PBW | 17 | 90.5 | 159.4 | 68.9 |
| CBG | 17 | 90.7 | 156.8 | 66.1 |
| CBR | 17 | 89.2 | 155.4 | 66.2 |
| CBW | 17 | 88.8 | 161.2 | 72.4 |
| CHG | 17 | 77.8 | 135 | 57.2 |
| CHR | 16 | 74.8 | 134.5 | 59.7 |
| CHW | 17 | 82.0 | 141.3 | 59.3 |

P = PURCHASED
C = C_DAIRY
B = BULL CALF
H = HEIFER CALF
G = POSITIVE CONTROL with liquid product
R = NEGATIVE CONTROL
W = TREATMENT with freeze dried product

Texas Calf Treatment Trial

The objective of this study was to compare 3 different treatments for calves. To ensure that each treatment was randomly assigned the treatment was assigned to the calves in the order they were born. Both bull calves and heifer calves were treated. Each calf was assigned to the treatment group according to the color of the card the calf's number appeared on. The cards were printed on three different color card stock. The assignment of the treatment used for each treatment group was:
Pink Calf card—Red Powder Treatment—Negative Control
White Calf card—White Powder Treatment—Warm Air Dried Positive Control
Green Calf card—Green Liquid Treatment—Treatment Group

The calf's dam's number and birth date were recorded on the cards. The workers were asked to check the appropriate space if the calf is a twin or if the cow needed assistance to deliver the calf (the calf was pulled).

The treatment assigned was given for seven days. The calves were treated only 1 time per day in the morning. The calf was to receive colostrum the first day and treatment for the next 7 consecutive days. The calf feeder was asked to circle the day of birth and then X each day the treatment is given.

The mixing and feeding instructions given to the calf feeders were:

Mix the treatment in the milk prior to feeding the calf. The treatment may be mixed for several calves at once, however it may tend to settle out if allowed to stand. The bottles should be filled immediately after mixing the treatment and then inverted once or twice prior to feeding. If the milk has to stand in a five-gallon container following mixing prior to feeding or pouring into bottles, remix the container prior to pouring up for the calves. Once mixed the milk will have a color the same as the card. Pink milk to the calves with a pink card, white milk (grayish color) to the calves with white cards and green milk to calves with a green card.

The two powder treatment are mixed as 2 level teaspoons (tsp—small spoon) per bottle. When mixing for several calves, mix 6 Tablespoons (tblsp—large spoon) per 5-gallon bucket.

The liquid treatment is mixed at the rate of 8 cc per bottle or 80 cc per 5-gallon bucket. Shake well before drawing out this medicine.

Although the treatment is only given for seven days, the effects are expected to last until weaning. The calves should be monitored daily until weaning or until the individual pages are collected (this may be done prior to weaning if the calves appear normal).

The monitoring instructions used during this trial are as follows:
Daily—Record the score of the manure from the calf. The scores to be used are:
1. Normal (1)—Firm but not hard. Original form is distorted slightly after dropping to floor and settling.
2. Soft (2)—Does not hold form, piles but spreads slightly. Similar to soft serve ice cream.
3. Runny (3)—Spreads readily to about ¼ of an inch (6 mm) in depth. Similar to pancake batter.
4. Watery (4)—Liquid consistency, splatters. Similar to orange juice.

If there is some question as to whether the manure is one score or another, for example: soft or runny, just list both scores for that day. If diarrhea develops during the day, simply write in the second score with PM after it for the later observation. If diarrhea continues for 4 days and it is watery for the four days this should be recorded each day as 4. An example of the records follows. In the example the first day (November 1) was normal and this is recorded as a 1. The second day (November 2) the calf had soft manure in the morning and watery diarrhea in the afternoon. This would be recorded as a 2 for the soft manure in the morning and as a 4 followed by PM for the watery manure in the afternoon. The next three days the calf has watery diarrhea (Nov. 3-5, and recorded as a 4). The calf is better on November 6 and the manure is not runny but really isn't firm enough to be soft. This would be recorded as a 2 for soft and a 3 for runny. On November 7 the calf is headed for recovery and the manure is soft recorded as a 2.

| Nov 1 | Nov 2 | Nov 3 | Nov 4 | Nov 5 | Nov 6 | Nov 7 |
|---|---|---|---|---|---|---|
| 1 | 2 | 4 | 4 | 4 | 2-3 | 2 |
| | 4 PM | | | | | |

Treatment descriptions are:
Green Liquid Treatment—Autoclaved liquid rumen fluid
Red Powder Treatment—Milk powder with red Kool-Aid®
White Powder Treatment—Warm Air Dried Rumen Fluid on ground rice base with added probiotics, vitamins and trace minerals.
   Results:

There were no differences in manure consistency scores between treatments. The number of antibiotic treatments administered to animals for diarrhea was reduced by 50% for treated calves. There were no deaths of treated calves but 4 and 3 deaths in the two control groups. No body weights were recorded in this trial.

Texas Calf Trial

| | | # antibiotic | | # animals |
|---|---|---|---|---|
| | # calves | treatments | | treated with |
| Treatment | per treatment | for group | # Deaths | antibiotics |
| GH | 12 | 9 | 0 | 6 |
| RH | 13 | 12 | 3 | 5 |
| WH | 13 | 14 | 2 | 7 |
| GB | 14 | 1 | 0 | 1 |
| RB | 13 | 8 | 1 | 5 |
| WB | 11 | 9 | 1 | 6 |
| Total G | 26 | 10 | 0 | 7 |
| Total R | 26 | 20 | 4 | 10 |
| Total W | 24 | 23 | 3 | 13 |

G = Liquid product treatment
R = Negative control
W = Heat dried positive control
B = Bull calf
H = Heifer calf
WB - Two of these calves died within 24 hours following birth

I claim:

1. A method for the promotion of growth and weight gain, the abatement of diarrheal disease and the reduction in mortality in a farm animal comprising the administration of bacterial polysaccharides derived from dried rumen fluid combined with nutritional aids for the first 1-7 days of life.

2. A method according to claim 1, in which the farm animal is a calf.

3. A method according to claim 1, in which the farm animal is a pig.

4. A method according to claim 1, in which the farm animal is a chick.

5. A method according to claim 1, in which the farm animal is a turkey poult.

6. A method according to claim 1, in which the farm animal is a foal.

7. A method according to claim 1, in which the farm animal is a kid.

8. A method according to claim 1, in which the farm animal is a lamb.

9. A method according to claim 1, in which the nutritional aids contain 20-95% of the amino acid threonine.

10. A method according to claim 1, in which the nutritional aids contain 5-70% of a monosaccharide.

11. A method according to claim 1, in which the nutritional aids contain the recommended daily dose of vitamins A, D, E for the newborn of the species being fed.

12. A method according to claim 1, in which the nutritional aids contain a specially selected probiotic for the species being fed.

13. A method according to claim 1, in which the nutritional aids contain the recommended daily dose of Thiamine, Riboflavin, Pyridoxine, Pantothenic acid, Niacin, Biotin, Folic acid, B₁₂ and Choline for the newborn of the species being fed.

14. A method according to claim 1, in which the nutritional aids contain the organic trace minerals Manganese, Zinc, Copper, and Selenium.

15. A preventative feed additive composition for the promotion of growth and weight gain, the abatement of diarrheal disease and the reduction in mortality in a farm animal comprising the administration of bacterial polysaccharides derived from dried rumen fluid combined with nutritional aids for the first 1-7 days of life.

16. The composition in claim 15, wherein the nutritional aids contains 20-95% of the amino acid threonine.

17. The composition in claim 15, wherein the nutritional aids contains 5-70% of a monosaccharide.

18. The composition in claim 15, wherein the nutritional aids contain the recommended daily dose of vitamins A, D, E for the newborn of the species being fed.

19. The composition in claim 15, wherein the nutritional aids contains a specially selected probiotic for the species being fed.

20. The composition in claim 15, wherein the nutritional aids contain the recommended daily dose of Thiamine, Riboflavin, Pyridoxine, Pantothenic acid, Niacin, Biotin, Folic acid, B₁₂ and Choline for the newborn of the species being fed.

21. The composition in claim 15, wherein the nutritional aids contains the organic trace minerals Manganese, Zinc, Copper, and Selenium.

22. A preventative feed additive composition in claim 15, in which the intended farm animal is a calf.

23. A preventative feed additive composition in claim 16, in which the intended farm animal is a calf.

24. A preventative feed additive composition in claim 17, in which the intended farm animal is a calf.

25. A preventative feed additive composition in claim 18, in which the intended farm animal is a calf.

26. A preventative feed additive composition in claim 19, in which the intended farm animal is a calf.

27. A preventative feed additive composition in claim 20, in which the intended farm animal is a calf.

28. A preventative feed additive composition in claim 21 in which the intended farm animal is a calf.

29. A preventative feed additive composition in claim 15 in which the intended farm animal is a pig.

30. A preventative feed additive composition in claim 16 in which the intended farm animal is a pig.

31. A preventative feed additive composition in claim 17 in which the intended farm animal is a pig.

32. A preventative feed additive composition in claim 18 in which the intended farm animal is a pig.

33. A preventative feed additive composition in claim 19 in which the intended farm animal is a pig.

34. A preventative feed additive composition in claim 20 in which the intended farm animal is a pig.

35. A preventative feed additive composition in claim 21 in which the intended farm animal is a pig.

36. A preventative feed additive composition in claim 15 in which the intended farm animal is a chick.

37. A preventative feed additive composition in claim 16 in which the intended farm animal is a chick.

38. A preventative feed additive composition in claim 17 in which the intended farm animal is a chick.

39. A preventative feed additive composition in claim 18 in which the intended farm animal is a chick.

40. A preventative feed additive composition in claim 19 in which the intended farm animal is a chick.

41. A preventative feed additive composition in claim 20 in which the intended farm animal is a chick.

42. A preventative feed additive composition in claim 21 in which the intended farm animal is a chick.

43. A preventative feed additive composition in claim 15 in which the intended farm animal is a foal.

44. A preventative feed additive composition in claim 16 in which the intended farm animal is a foal.

45. A preventative feed additive composition in claim 17 in which the intended farm animal is a foal.

46. A preventative feed additive composition in claim 18 in which the intended farm animal is a foal.

47. A preventative feed additive composition in claim 19 in which the intended farm animal is a foal.

48. A preventative feed additive composition in claim 20 in which the intended farm animal is a foal.

49. A preventative feed additive composition in claim 21 in which the intended farm animal is a foal.

50. A preventative feed additive composition in claim 15 in which the intended farm animal is a turkey poult.

51. A preventative feed additive composition in claim 16 in which the intended farm animal is a turkey poult.

52. A preventative feed additive composition in claim 17 in which the intended farm animal is a turkey poult.

53. A preventative feed additive composition in claim 18 in which the intended farm animal is a turkey poult.

54. A preventative feed additive composition in claim 19 in which the intended farm animal is a turkey poult.

55. A preventative feed additive composition in claim 20 in which the intended farm animal is a turkey poult.

56. A preventative feed additive composition in claim 21 in which the intended farm animal is a turkey poult.

57. A preventative feed additive composition in claim 15 in which the intended farm animal is a kid.

58. A preventative feed additive composition in claim 16 in which the intended farm animal is a kid.

59. A preventative feed additive composition in claim 17 in which the intended farm animal is a kid.

60. A preventative feed additive composition in claim 18 in which the intended farm animal is a kid.

61. A preventative feed additive composition in claim 19 in which the intended farm animal is a kid.

62. A preventative feed additive composition in claim 20 in which the intended farm animal is a kid.

63. A preventative feed additive composition in claim 21 in which the intended farm animal is a kid.

64. A preventative feed additive composition in claim 15 in which the intended farm animal is a lamb.

65. A preventative feed additive composition in claim 16 in which the intended farm animal is a lamb.

66. A preventative feed additive composition in claim 17 in which the intended farm animal is a lamb.

67. A preventative feed additive composition in claim 18 in which the intended farm animal is a lamb.

68. A preventative feed additive composition in claim 19 in which the intended farm animal is a lamb.

69. A preventative feed additive composition in claim 20 in which the intended farm animal is a lamb.

70. A preventative feed additive composition in claim 21 in which the intended farm animal is a lamb.

71. A method for collecting, processing, sterilizing and stabilizing rumen fluid to retain the bioactivity of the bacterial polysaccharides found therein.

72. A method of freeze-drying sterilized rumen to maintain the bioactivity of the bacterial polysaccharides.

73. A method of synthesizing rumen bacterial polysaccharides in increased amounts using a cow as the growth apparatus, a special formulation of normal cow feed as a substrate and a combination of feeding time, time of day, feeding technique, collection area, feed physical form and number of feedings to maximize bacterial growth and diversity.

74. A method of standardizing the dose of bacterial polysaccharides by measuring the amount of bacterial polysaccharides in the rumen fluid, measuring the amount of fluid dried, then determining the total amount of bacterial polysaccharides contained in the dried rumen fluid wafer. This quantity of bacterial polysaccharides is then used to determine the total number of doses that may be produced. The freeze dried rumen fluid wafer is then weighed and an amino acid carrier (threonine) is added to Q. S. the total to the weight represented by the weight of the total number of doses that may be produced.
